(19) United States
(12) Patent Application Publication
(10) Pub. No.: US 2021/0259381 A1
(43) Pub. Date: Aug. 26, 2021

(54) PROCESS FOR CURLING KERATIN FIBRES COMPRISING THE APPLICATION TO THE FIBRES OF A COMPOSITION CONTAINING THIOLACTIC ACID

(71) Applicant: L'OREAL, Paris (FR)

(72) Inventors: Laëtitia Feuillette, Clichy (FR); Estelle Millet, Saint Ouen (FR); Gaëlle Calonne, Saint Ouen (FR)

(73) Assignee: L'OREAL, Paris (FR)

(21) Appl. No.: 17/256,644

(22) PCT Filed: Jun. 28, 2019
(86) PCT No.: PCT/EP2019/067442

§ 371 Date: Dec. 28, 2020

(30) Foreign Application Priority Data
Jun. 28, 2018 (FR) 1855852

(51) Int. Cl. A45D 7/06; A61K 8/46; A61Q 5/06

(52) U.S. Cl. CPC A45D 7/06; A61K 2800/48; A61Q 5/06; A61K 8/46

(57) ABSTRACT

The present invention relates to a process for curling keratin fibres, such as the hair, which comprises at least the rolling of said fibres around one or more curling means, the application of a reductive acidic cosmetic composition, and a step of heating said fibres. The invention also relates to the use of said cosmetic composition for curling keratin fibres which have been dyed and/or lightened beforehand.

The present invention relates to a process for curling keratin fibres, such as the hair, which comprises at least the rolling of said fibres around one or more curling means, the application of a reductive acidic cosmetic composition, and a step of heating said fibres.

A subject of the invention is also the use of said cosmetic composition for curling keratin fibres which have been dyed and/or lightened beforehand.

Many people are not satisfied with the appearance of their hair. In particular, people who have straight hair usually wish to obtain hair with beautiful curls and good volume.

To obtain curling of the hair, the technique most commonly used consists, in a first stage, in opening the —S—S— disulfide bonds of keratin (keratocystine) by means of a generally basic composition containing a sulfur-based reducing agent (reduction step), and then, after having rinsed the head of hair thus treated, generally with water, in reconstituting, in a second stage, said disulfide bonds by applying to the hair, which has been placed under tension beforehand, an oxidizing composition (oxidation step, also known as the fixing step) so as finally to give the hair the desired shape.

The new shape given to the hair by such a chemical treatment is eminently long-lasting and notably withstands washing with water or shampoos, as opposed to the simple standard techniques of temporary reshaping.

Many products intended for curling exist on the market.

The products intended for curling are generally formulated either using alkaline compositions, with a pH above 8.5, and/or using a high concentration of thiols, such as mercaptan compounds.

The application of these products requires precise know-how, which is mainly due to the high contents of reducing agents used in the reducing compositions or to the high contents of hydroxides and/or to the very alkaline pH of the curling compositions, and also to the various longer or shorter leave-on times of these compositions.

It has also been found that the use of these reducing agents or of these strong alkaline agents is not entirely satisfactory in terms of protection and integrity of the fibre on sensitized hair, i.e. hair that is generally damaged or embrittled by the action of external atmospheric agents, such as light and bad weather, and/or mechanical or chemical treatments, such as brushing, combing, dyeing, bleaching, permanent waving and/or relaxing and/or heat treatments.

More particularly, it has been found that the known hair curling processes performed on hair which has undergone colouring and/or lightening operations beforehand make the hair more brittle and lead to an increase in the lengthening of the hair. Moreover, the cosmetic properties are generally degraded, in particular in terms of the quality of the feel (for example softness) and/or the suppleness.

There is thus a real need to develop processes for curling keratin fibres that are capable of reducing the damage caused to keratin fibres, and also of preserving their integrity, their mechanical properties, and the cosmetic properties notably for keratin fibres which have undergone colouring and/or lightening operations beforehand. Such processes must also make it possible to obtain beautiful, well-defined hair curls, with good volume of the head of hair and hair with good cosmetic working properties, notably good softness of touch and good suppleness, and to do so in a long-lasting manner.

These aims are achieved by the present invention, one subject of which is notably a process for curling keratin fibres, in particular human keratin fibres such as the hair, comprising at least the following steps:
i) rolling said fibres around one or more keratin fibre curling means;
ii) applying to said fibres an aqueous cosmetic composition:
   a. comprising at least one reducing agent chosen from thiolactic acid, salts thereof, and mixtures thereof, and
   b. with a pH between 2.5 and 6;
iii) heating said fibres to a temperature of greater than or equal to 35° C.; it being understood that step iii) is performed after steps i) and ii).

It has notably been found that the process according to the invention makes it possible to obtain beautiful, well-defined hair curls and good hair volume.

The hair curls obtained via the process of the invention show good persistence with respect to shampoo washing; in particular, the hair curls obtained withstand at least 5 shampoo washes, or even at least 10 shampoo washes.

It has also been found that hair treated according to the process of the invention, notably hair which has undergone colouring and/or lightening operations beforehand, shows good fibre integrity and conserves good mechanical properties, for example with respect to the fibre elasticity.

The hair curls obtained via the process of the invention also have good suppleness and good softness of touch.

A subject of the invention is also the use of the cosmetic composition according to the invention for curling keratin fibres which have been dyed and/or lightened beforehand.

Other subjects, characteristics, aspects and advantages of the invention will emerge even more clearly on reading the description and the example that follows.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;
for the purposes of the present invention, the expression “less than” and, respectively, the expression “greater than” mean an open range which is strictly less, or, respectively, strictly greater, and therefore that the limits are not included;
according to the present patent application, the term “keratin fibres” denotes human keratin fibres and more particularly the hair.

The Keratin Fibre Curling Means

The process according to the invention comprises at least one step of rolling keratin fibres around one or more keratin fibre curling means.

More particularly, during step i) of the process according to the invention, the keratin fibres are placed under mechanical tension using a keratin fibre curling means.

Preferably, the keratin fibre curling means are chosen from curlers and heated curlers.

According to this preference, the diameter and length of the curling means are chosen as a function of the desired keratin fibre curl size.

More preferentially, the diameter of the curlers and/or heated curlers ranges from 0.5 to 3 cm, better still from 0.7 to 2 cm; and/or the length of the curlers and/or heated curlers ranges from 3 to 10 cm, better still from 4 to 8 cm.

The Cosmetic Composition

The process according to the invention comprises at least one step of applying to said fibres a cosmetic composition:
a. comprising at least one reducing agent chosen from thiolactic acid, salts thereof, and mixtures thereof, and
b. with a pH between 2.5 and 6.

The Reducing Agents

The cosmetic composition according to the invention comprises at least one reducing agent chosen from thiolactic acid, salts thereof, and mixtures thereof.

Preferably, the composition according to the invention comprises at least thiolactic acid.

Preferably, the total content of reducing agent(s) chosen from thiolactic acid, salts thereof, and mixtures thereof, present in the composition according to the invention, is between 1% and 15% by weight, more preferentially between 2% and 13% by weight, even more preferentially between 3% and 12%, even better still between 4% and 10% by weight, relative to the total weight of the composition.

Preferably, the content of thiolactic acid present in the composition according to the invention is between 1% and 15% by weight, more preferentially between 2% and 13% by weight, even more preferentially between 3% and 12%, even better still between 4% and 10% by weight, relative to the total weight of the composition.

Preferably, the composition according to the invention comprises one or more additional agents chosen from one or more nonionic surfactants, one or more cationic surfactants; one or more anionic surfactants, one or more thickening polymers, one or more silicones, one or more alkaline agents, and/or one or more non-silicone fatty substances.

The Nonionic Surfactants

The cosmetic composition according to the invention may also optionally comprise one or more nonionic surfactants.

They may be chosen from alcohols, α-diols and (C₁₋₂₀)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or alternatively these compounds comprising at least one fatty chain including from 8 to 30 carbon atoms and notably from 16 to 30 carbon atoms.

Mention may also be made of condensates of ethylene oxide and of propylene oxide with fatty alcohols; polyethoxylated fatty amides preferably containing from 2 to 30 ethylene oxide units, polyglycerolated fatty amides including on average from 1 to 5, and in particular from 1.5 to 4, glycerol groups; ethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

Mention may also be made of nonionic surfactants of alkyl(poly)glycoside type, represented notably by the following general formula:

R₁O—(R₂O)ₜ-(G)ᵥ

in which:
R₁ represents a linear or branched alkyl or alkenyl radical including 6 to 24 carbon atoms and notably 8 to 18 carbon atoms, or an alkylphenyl radical of which the linear or branched alkyl radical includes 6 to 24 carbon atoms and notably 8 to 18 carbon atoms,
R₂ represents an alkylene radical including 2 to 4 carbon atoms,
G represents a sugar unit including 5 to 6 carbon atoms,
t denotes a value ranging from 0 to 10 and preferably from 0 to 4,
v denotes a value ranging from 1 to 15 and preferably from 1 to 4.

Preferably, the alkyl(poly)glycoside surfactants are compounds of the formula described above in which:
R₁ denotes a linear or branched, saturated or unsaturated alkyl radical including from 8 to 18 carbon atoms,
R₂ represents an alkylene radical including 2 to 4 carbon atoms,
t denotes a value ranging from 0 to 3 and preferably equal to 0,
G denotes glucose, fructose or galactose, preferably glucose,
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C8/C16 Alkyl(poly)glucosides 1,4, and in particular decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or the products sold by the company Chem Y under the name AG10 LK.

Preferably, use is made of C₈/C₁₆-alkyl (poly)glycosides 1,4, notably as an aqueous 53% solution, such as those sold by Cognis under the reference Plantacare® 818 UP.

Preferably, the cosmetic composition according to the invention comprises one or more nonionic surfactants; preferentially chosen from (C₆-C₂₄ alkyl) (poly)glycosides, and more particularly (C₈-C₁₈alkyl)(poly)glycosides, ethoxylated C₈-C₃₀ fatty acid esters of sorbitan, polyethoxylated C₅-C₃₀ fatty alcohols, polyoxyethylenated C₅-C₃₀ fatty acid esters, these compounds preferably containing 2 to 150 mol of ethylene oxide, and mixtures thereof; even more preferentially from (C₈-C₁₈ alkyl)(poly)glycosides, ethoxylated C₅-C₃₀ fatty acid esters of sorbitan, polyethoxylated C₅-C₃₀ fatty alcohols notably containing from 2 to 150 mol of ethylene oxide, and mixtures thereof.

Preferably, when the nonionic surfactant(s) are present in the composition according to the invention, the total content of nonionic surfactant(s) present is between 0.01% and 20% by weight, more preferentially between 0.1% and 15% by weight, even more preferentially between 0.2% and 10% by weight, even better still between 0.5% and 6% by weight, relative to the total weight of the composition.

The Cationic Surfactants

The cosmetic composition according to the invention may also optionally comprise one or more cationic surfactants.

They are advantageously chosen from optionally polyoxyalkylenated primary, secondary or tertiary fatty amine salts, quaternary ammonium salts, and mixtures thereof.

As quaternary ammonium salts, mention may be made notably of:
the quaternary ammonium salts of formula (Ia):

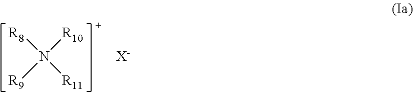

in which:
the groups R₈ to R₁₁, which may be identical or different, represent a linear or branched aliphatic group including from 1 to 30 carbon atoms, or an aromatic group such as aryl or alkylaryl, at least one of the groups R₈ to R₁ including from 8 to 30 and preferably from 12 to 24 carbon atoms, it being possible for the aliphatic groups to include heteroatoms notably such as oxygen, nitrogen, sulfur and halogens; and
X⁻ is an anion chosen notably from the group of halides, phosphates, acetates, lactates, (C₁-C₄)alkyl sulfates, (C₁-C₄)alkylsulfonates or (C₁-C₄)alkylarylsulfonates.

The aliphatic groups R₈ to R₁₁ may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Mention may be made notably of tetraalkylammonium halides, notably chlorides, such as dialkyldimethylammonium or alkyltrimethylammonium chlorides in which the alkyl group includes from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and notably chlorides; notably the product sold under the name Ceraphyl® 70 by the company Van Dyk.
the quaternary ammonium salts of imidazoline of formula (Ia):

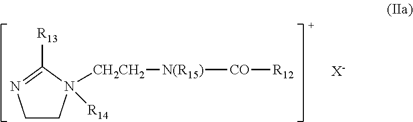

in which
R₁₂ represents an alkenyl or alkyl group including from 8 to 30 carbon atoms, for example derived from tallow fatty acids,
R₁₃ represents a hydrogen atom, a C₁-C₄ alkyl group or an alkenyl or alkyl group including from 8 to 30 carbon atoms,
R₁₄ represents a C₁-C₄ alkyl group,
R₁₅ represents a hydrogen atom or a C₁-C₄ alkyl group,
X⁻ is an anion chosen notably from the group of the halides, phosphates, acetates, lactates, (C₁-C₄)alkyl sulfates, and (C₁-C₄)alkylsulfonates or (C₁-C₄)alkylarylsulfonates.

Preferably, R₁₂ and R₁₃ denote a mixture of alkenyl or alkyl groups including from 12 to 21 carbon atoms, for example derived from tallow fatty acids, R₁₄ denotes a methyl group and R₁₅ denotes a hydrogen atom. Such a product is sold, for example, under the name Rewoquat® W75 or W90 by the company Evonik.
the quaternary di- or triammonium salts of formula (IIIa):

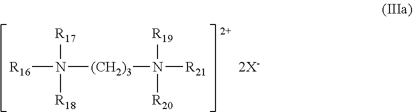

in which:
R₁₆ denotes an alkyl group including from 16 to 30 carbon atoms, which is optionally hydroxylated and/or optionally interrupted with one or more oxygen atoms,
R₁₇ denotes hydrogen, an alkyl group including from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group including from 1 to 4 carbon atoms,
R₁₈, R₁₉, R₂₀ and R₂₁, which may be identical or different, denote hydrogen or an alkyl group including from 1 to 4 carbon atoms, and
X⁻ is an anion, chosen notably from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P (Quaternium 89) and Finquat CT (Quaternium 75), sold by the company Finetex.
quaternary ammonium salts containing one or more ester functions, of formula (IVa) below:

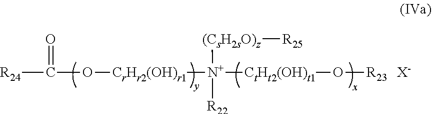

in which:
R₂₂ is chosen from C₁-C₆ alkyl groups and C₁-C₆ hydroxyalkyl or dihydroxyalkyl groups,
R₂₃ is chosen from the group R₂₆—C(═O)—; linear or branched, saturated or unsaturated C₁-C₂₂ hydrocarbon-based groups R₂₇; and a hydrogen atom,
R₂₅ is chosen from the group R₂₈—C(═O)—; linear or branched, saturated or unsaturated C₁-C₆ hydrocarbon-based groups R₂₉; and a hydrogen atom,
R₂₄, R₂₆ and R₂₈, which may be identical or different, are chosen from linear or branched, saturated or unsaturated C₇-C₂₁ hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻ is an anion,
it being understood that r2+r1=2r and t1+t2=2t, and that the sum x+y+z is from 1 to 15,
with the proviso that when x=0 then R₂₃ denotes R₂₇ and that when z=0 then R₂₅ denotes R₂₉.

The alkyl groups R₂₂ may be linear or branched, preferably linear. Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may comprise from 12 to 22 carbon atoms, or else may comprise from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate, a (C₁-C₄)alkylsulfonate or a (C₁-C₄)alkylarylsulfonate, a methanesulfonate, a phosphate, a nitrate, a tosylate, an anion derived from organic acid such as an acetate or a lactate or any other anion that is compatible with the ammonium bearing an ester function. The anion X⁻ is more particularly a chloride, a methyl sulfate or an ethyl sulfate.

Use is more particularly made, in the composition according to the invention, of the ammonium salts of formula (IVa) in which:
R₂₂ denotes a methyl or ethyl group,
x and y are equal to 1,
z is equal to 0 or 1,
r, s and t are equal to 2,
R₂₃ is chosen from the group R₂₆—C(═O)—; methyl, ethyl or C₁₄-C₂₂ hydrocarbon-based groups, and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (IVa), mention may be made of the salts, notably the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures notably of plant or animal origin, or by transesterification of the methyl esters thereof. This esterification may be followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a weight majority of diester salts. Use may also be made of the ammonium salts containing at least one ester function that are described in patents U.S. Pat. Nos. 4,874,554 and 4,137,180. Use may also be made of the behenoylhydroxypropyltrimethylammonium chloride sold, for example, by the company Kao under the name Quartamin BTC 131.

Preferably, the ammonium salts containing at least one ester function contain two ester functions.

According to a particular embodiment, the cosmetic composition according to the invention comprises one or more cationic surfactants; preferentially chosen from those of formula (Ia) or (IVa), even more preferentially from cetyltrimethylammonium salts, behenyltrimethylammonium salts and dipalmitoylethylhydroxyethylmethylammonium salts, and mixtures thereof; and even better still from behenyltrimethylammonium chloride or methosulfate, cetyltrimethylammonium chloride or methosulfate, dipalmitoylethylhydroxyethylmethylammonium chloride or methosulfate, and mixtures thereof.

Preferably, when the cationic surfactant(s) are present in the composition according to the invention, the total content of cationic surfactant(s) present is between 0.1% and 10% by weight, more preferentially between 0.5% and 8% by weight, even more preferentially between 1% and 50% by weight, relative to the total weight of the composition.

Anionic Surfactants:

The cosmetic composition according to the invention may also optionally comprise one or more anionic surfactants.

The term “anionic surfactant” means a surfactant including, as ionic or ionizable groups, only anionic groups.

In the present description, a species is termed “anionic” when it bears at least one permanent negative charge or when it can be ionized into a negatively charged species, under the conditions of use of the composition of the invention (for example the medium or the pH) and not comprising any cationic charge.

The anionic surfactants may be sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylic anionic surfactants that may be used thus comprise at least one carboxylic or carboxylate function (—COOH or —COO⁻).

The anionic surfactants may be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds;

the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (1):

R₁—(OC₂H₄)ₙ—OCH₂COOA (1)

in which:
R1 represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;

preferably, R₁ is a C₅-C₂₀ and preferably C₈-C₁₈alkyl radical, and aryl preferably denotes phenyl,
n is an integer or decimal number (average value) ranging from 2 to 24 and preferably from 2 to 10,
A denotes H, ammonium, Na, K, Li, Mg or a monoethanolamine or triethanolamine residue.

It is also possible to use mixtures of compounds of formula (1), in particular mixtures of compounds containing different groups R1.

The polyoxyalkylenated alkyl(amido) ether carboxylic acids that are particularly preferred are those of formula (1) in which:
R₁ denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (1) in which R denotes a C₁₂ alkyl radical, A denotes a hydrogen or sodium atom and n ranges from 2 to 10.

Preferentially, the carboxylic anionic surfactants are chosen, alone or as a mixture, from:
acylglutamates, notably of C₆-C₂₄ or even C₁₂-C₂₀, such as stearoylglutamates, and in particular disodium stearoylglutamate;
acylsarcosinates, notably of C₆-C₂₄ or even C₁₂-C₂₀, such as palmitoylsarcosinates, and in particular sodium palmitoylsarcosinate;
acyllactylates, notably of C₁₂-C₂₈ or even C₁₄-C₂₄, such as behenoyllactylates, and in particular sodium behenoyllactylate;
C₆-C₂₄ and notably C₁₂-C₂₀ acylglycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;

in particular in the form of alkali metal or alkaline-earth metal, ammonium or amino alcohol salts.

The sulfonate anionic surfactants that may be used include at least one sulfonate function (—SO₃H or —SO₃⁻).

They may be chosen from the following compounds: alkylsulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, in particular polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acylisethionates;

in particular in the form of alkali metal or alkaline-earth metal, ammonium or amino alcohol salts.

The sulfate anionic surfactants that may be used include at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Preferentially, the sulfate anionic surfactants are chosen, alone or as a mixture, from:
alkyl sulfates, notably C₆-C₂₄ or even C₁₂-C₂₀ alkyl sulfates;
alkyl ether sulfates, notably C₆-C₂₄ or even C₁₂-C₂₀ alkyl ether sulfates, preferably comprising from 2 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferably, the anionic surfactants are chosen, alone or as a mixture, from:
C₆-C₂₄ and notably C₁₂-C₂₀ alkyl sulfates,
C₆-C₂₄ and notably C₁₂-C₂₀ alkyl ether sulfates; preferably comprising from 2 to 20 ethylene oxide units;
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acylisethionates;
C₆-C₂₄ and notably C₁₂-C₂₀ acylsarcosinates; notably palmitoylsarcosinates;
(C₆-C₂₄)alkyl ether carboxylates, preferably (C₁₂-C₂₀)alkyl ether carboxylates;
polyoxyalkylenated (C₆-C₂₄)alkyl(amido) ether carboxylic acids and salts thereof, in particular those including from 2 to 50 alkylene oxide and in particular ethylene oxide groups;
C₆-C₂₄ and notably C₁₂-C₂₀ acylglutamates;
C₆-C₂₄ and notably C₁₂-C₂₀ acylglycinates;

in particular in the form of alkali metal or alkaline-earth metal, ammonium or amino alcohol salts.

Among the anionic surfactants that may be used according to the invention, mention may also be made of phosphoric surfactants.

The term “phosphoric surfactant” means a surfactant whose polar part comprises at least one phosphorus atom.

The phosphoric surfactant may have the following formula:

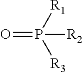

in which

R₁, R₂ and R₃, which may be identical or different, represent a group chosen from:
a group —OM, in which M represents a hydrogen atom or an alkali metal, such as Na, Li or K, preferably Na or K;
a group —OR₄, in which R₄ represents a linear or branched C₁-C₄₀ alkyl group, preferably a C₁₂-C₂₀ alkyl group and more preferably a C₁₆ or C₁₈ alkyl group, a linear or branched C₂-C₄₀ alkenyl group, preferably a C₁₂-C₂₀ alkenyl group and more preferably a C₁₆ or C₁₈ alkenyl group, a C₃-C₄₀ cyclic alkyl group, a C₃-C₄₀ cyclic alkenyl group, a C₅-C₄₀ aromatic group or a C₆-C₄₀ aralkyl group; and
an oxyalkylene group —(OCH₂CH₂)ₙ(OCH₂CH(CH₃))ₘOR₄ in which R₄ is as defined previously, n represents an integer ranging from 1 to 50 and m represents an integer ranging from 0 to 50,

given that at least one from among R₁, R₂ and R₃ is a group —OM and that at least one from among R₁, R₂ and R₃ is a group —OR₄ or —(OCH₂CH₂)ₙ(OCH₂CH(CH₃))ₘOR₄.

Preferably, the phosphoric surfactant may be chosen from oxyalkylenated C₁₂-C₂₀ alcohol phosphates containing from 1 to 50 mol of alkylene oxide chosen from ethylene oxide and propylene oxide, and non-oxyalkylenenated C₁₂-C₂₀ alcohol dialkyl phosphates, and mixtures thereof. The alkyl groups of the oxyalkylenated alcohol and/or of the non-oxyalkylenated alcohol may be linear or branched, and saturated or unsaturated.

Preferably, use may be made of a combination of at least one oxyalkylenated phosphoric surfactant and of at least one non-oxyalkylenated phosphoric surfactant.

More preferably, the combination of phosphoric surfactants may be chosen from the group consisting of a combination of ceteth-10 phosphate and dicetyl phosphate, a combination of ceteth-20 phosphate and dicetyl phosphate, and a combination of oleth-5 phosphate and dioleyl phosphate.

As product comprising the combination of ceteth-10 phosphate and dicetyl phosphate, mention may be made of Crodafos CES or Crodafos CES-PA, sold by Croda. As product comprising the combination of ceteth-20 phosphate and dicetyl phosphate, mention may be made of Crodafos CS-20 Acid, sold by Croda. As product comprising the combination of oleth-5 phosphate and dioleyl phosphate, mention may be made of Crodafos HCE, sold by Croda.

According to a preferred embodiment of the invention, the cosmetic composition comprises one or more anionic surfactants; more preferentially, the anionic surfactant(s) are chosen from oxyalkylenated C₁₂-C₂₀ alcohol phosphates containing from 1 to 50 mol of alkylene oxide, non-oxyalkylenated C₁₂-C₂₀ alcohol dialkyl phosphates, and mixtures thereof.

Preferably, when the anionic surfactant(s) are present in the composition according to the invention, the total content of anionic surfactant(s) present is between 0.1% and 10% by weight, more preferentially between 0.5% and 8% by weight, even more preferentially between 1% and 50% by weight, relative to the total weight of the composition.

The Thickening Polymers

The cosmetic composition according to the invention may also optionally comprise one or more thickening polymers.

According to the present invention, the term “thickening polymers” means polymers which, by their presence at a concentration of 0.05% by weight, increase the viscosity of the cosmetic compositions into which they are introduced by at least 20 cps (20 mPa·s), preferably by at least 50 cps (50 mPa·s), at room temperature (25° C.), at atmospheric pressure and at a shear rate of 1 s⁻¹ (the viscosity may be measured using a cone/plate viscometer, a Haake R600 rheometer or the like).

The thickening polymer(s) that may be used in the process according to the invention are preferentially chosen from non-associative thickening polymers bearing sugar units, non-associative thickening polymers without sugar units, associative thickening polymers, and mixtures thereof.

For the purposes of the present invention, the term “sugar unit” means an oxygen-bearing hydrocarbon-based compound containing several alcohol functions, with or without aldehyde or ketone functions, and which includes at least 4 carbon atoms.

The sugar units may be optionally modified by substitution, and/or by oxidation and/or by dehydration.

The sugar units of the thickening polymers are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

According to the invention, polymers bearing sugar units are also known as polysaccharides.

Non-associative thickening polymers bearing sugar units that may notably be mentioned include native gums such as:
a) tree or shrub exudates, including:
   gum arabic (branched polymer of galactose, arabinose, rhamnose and glucuronic acid);
   ghatti gum (polymer derived from arabinose, galactose, mannose, xylose and glucuronic acid);
   karaya gum (polymer derived from galacturonic acid, galactose, rhamnose and glucuronic acid);
   gum tragacanth (polymer of galacturonic acid, galactose, fucose, xylose and arabinose);
b) gums derived from algae, including:
   agar (polymer derived from galactose and anhydrogalactose);
   alginates (polymers of mannuronic acid and of glucuronic acid);
   carrageenans and furcellerans (polymers of galactose sulfate and of anhydrogalactose sulfate);
c) gums derived from seeds or tubers, including:
   guar gum (polymer of mannose and galactose);
   locust bean gum (polymer of mannose and galactose);
   fenugreek gum (polymer of mannose and galactose);
   tamarind gum (polymer of galactose, xylose and glucose);
   konjac gum (polymer of glucose and mannose);
d) microbial gums, including:
   xanthan gum (polymer of glucose, mannose acetate, mannose/pyruvic acid and glucuronic acid);
   gellan gum (polymer of partially acylated glucose, rhamnose and glucuronic acid);
   scleroglucan gum (glucose polymer);
e) plant extracts, including:
   cellulose (glucose polymer);
   starch (glucose polymer) and
   inulin.

These polymers may be physically or chemically modified. As physical treatment, mention may notably be made of the temperature.

Chemical treatments that may be mentioned include esterification, etherification, amidation and oxidation reactions. These treatments make it possible to produce polymers that may notably be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly)hydroxyalkyl groups.

Among the C₁-C₆ (poly)hydroxyalkyl groups, mention may be made, by way of example, of hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably varies from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules that may be used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified, notably by one or more of the following reactions: pregelatinization, oxidation, crosslinking, esterification, etherification, amidation, heat treatments.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) or Prejel 200 (gelatinized acetyl cassava distarch phosphate) by the company Avebe, or Structure Zea from National Starch (gelatinized corn distarch phosphate).

According to the invention, amphoteric starches may also be used, these amphoteric starches comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic. The cationic groups may be of primary, secondary, tertiary or quaternary amine type.

The starch molecules may be derived from any plant source of starch, notably such as corn, potato, oat, rice, tapioca, sorghum, barley or wheat. It is also possible to use the hydrolysates of the starches mentioned above. The starch is preferably derived from potato.

The non-associative thickening polymers of the invention may be cellulose-based polymers not including a C₁₀-C₃₀ fatty chain in their structure.

According to the invention, the term “cellulose-based polymer” refers to any polysaccharide compound having in its structure sequences of glucose residues linked together via β-1,4 bonds; in addition to unsubstituted celluloses, the cellulose derivatives may be anionic, cationic, amphoteric or nonionic.

Thus, the cellulose-based polymers that may be used according to the invention may be chosen from unsubstituted celluloses, including those in a microcrystalline form, and cellulose ethers.

Among these cellulose-based polymers, cellulose ethers, cellulose esters and cellulose ester ethers are distinguished.

Among the cellulose esters are mineral esters of cellulose (cellulose nitrates, sulfates, phosphates, etc.), organic esters of cellulose (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers without a C₁₀-C₃₀ fatty chain, i.e. which are “non-associative”, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl-(C₁-C₄)alkylcellulose celluloses, such as hydroxypropylmethylcelluloses (for example, Methocel E4M from Dow Chemical), hydroxyethylmethylcelluloses, hydroxyethylethylcelluloses (for example, Bermocoll E 481 FQ from AkzoNobel) and hydroxybutylmethylcelluloses.

Among the anionic cellulose ethers without a fatty chain, mention may be made of (poly)carboxy(C₁-C₄)alkylcelluloses and salts thereof. By way of example, mention may be made of carboxymethylcelluloses, carboxymethylmethylcelluloses (for example Blanose 7M from the company Aqualon) and carboxymethylhydroxyethylcelluloses, and the sodium salts thereof.

Among the cationic cellulose ethers without a fatty chain, mention may be made of cationic cellulose derivatives such as cellulose copolymers or cellulose derivatives grafted with a water-soluble quaternary ammonium monomer, and described notably in patent U.S. Pat. No. 4,131,576, such as (poly)hydroxy(C₁-C₄)alkylcelluloses, for instance hydroxymethyl-, hydroxyethyl- or hydroxypropylcelluloses grafted notably with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

Among the non-associative thickening polymers not bearing sugar units that may be used according to the invention, mention may be made of crosslinked acrylic acid or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or as mixtures.

A first family of non-associative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by the company Noveon or the products sold under the names Synthalen M and Synthalen K by the company 3 VSA. These polymers have the INCI name Carbomer.

The non-associative thickening polymers may also be crosslinked (meth)acrylic acid copolymers, such as the polymer sold under the name Aqua SF1 by the company Noveon.

The non-associative thickening polymers may be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of document EP 503853, and reference may be made to said document as regards these polymers.

The cosmetic composition may similarly comprise, as non-associative thickening polymers, ammonium acrylate homopolymers or copolymers of ammonium acrylate and of acrylamide.

Among the examples of ammonium acrylate homopolymers that may be mentioned is the product sold under the name Microsap PAS 5193 by the company Hoechst. Among the copolymers of ammonium acrylate and of acrylamide that may be mentioned is the product sold under the name Bozepol C Nouveau or the product PAS 5193 sold by the company Hoechst. Reference may be made notably to FR 2416 723, U.S. Pat. Nos. 2,798,053 and 2,923,692 as regards the description and preparation of such compounds.

Use may also be made of cationic thickening polymers of acrylic type.

Among the thickening polymers, mention may also be made of the associative polymers that are well known to a person skilled in the art and notably of nonionic, anionic, cationic or amphoteric nature.

It is recalled that “associative polymers” are polymers that are capable, in an aqueous medium, of reversibly associating with each other or with other molecules.

Their chemical structure more particularly comprises at least one hydrophilic region and at least one hydrophobic region.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type that may be mentioned are:
(a) those including at least one hydrophilic unit and at least one fatty-chain allyl ether unit, more particularly those of which the hydrophilic unit is constituted by an ethylenic unsaturated anionic monomer, even more particularly by a vinylcarboxylic acid and most particularly by an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), notably those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10);
(b) those including i) at least one hydrophilic unit of unsaturated olefinic carboxylic acid type, and ii) at least one hydrophobic unit of the (C₁₀-C₃₀) alkyl ester of an unsaturated carboxylic acid type.

(C₁₀-C₃₀) Alkyl esters of unsaturated carboxylic acids that are useful in the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to patents U.S. Pat. Nos. 3,915,921 and 4,509,949.

Among the anionic associative polymers of this type that will be used more particularly are those constituted of from 60% to 95% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those constituted of from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among said polymers above, the ones most particularly preferred according to the present invention are the products sold by the company Goodrich under the trade names Pemulen TR1®, Pemulen TR2®, Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP.
(c) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.
(d) acrylic terpolymers comprising:

i) about 20% to 70% by weight of an α,β-monoethylenically unsaturated carboxylic acid [A],

ii) about 20% to 80% by weight of an α,β-monoethylenically unsaturated non-surfactant monomer other than [A],

iii) about 0.5% to 60% by weight of a nonionic monourethane which is the product of reaction of a monohydric surfactant with a monoethylenically unsaturated monoisocyanate,

such as those described in patent application EP-A-0173109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(e) copolymers including among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas.
(f) amphiphilic polymers including at least one ethylenically unsaturated monomer bearing a sulfonic group, in free or partially or totally neutralized form and comprising at least one hydrophobic part. These polymers may be crosslinked or non-crosslinked. They are preferably crosslinked.

The ethylenically unsaturated monomers bearing a sulfonic group are notably chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used.

2-Acrylamido-2-methylpropanesulfonic acid (AMPS), and also partially or totally neutralized forms thereof, will more particularly be used.

The polymers of this family may be chosen notably from random amphiphilic AMPS polymers modified by reaction with a C₆-C₂₂ n-monoalkylamine or di-n-alkylamine, and such as those described in patent application WO 00/31154 (forming an integral part of the content of the description). These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not including a fatty chain, such as (meth)acrylic acids, 0-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described notably in patent application EP-A 750899, patent U.S. Pat. No. 5,089,578 and in the following publications from Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, 2000, Vol. 33, No. 10, 3694-3704;
Solution properties of micelle networks formed by nonionic moieties covalently bound to a polyelectrolyte: salt effects on rheological behavior—Langmuir, 2000 Vol. 16, No. 12, 5324-5332;
Stimuli responsive amphiphilic copolymers of sodium 2-(acrylamido)-2-methylpropanesulfonate and associative macromonomers, Polym. Preprint, Div. Polym. Chem., 40(2), (1999), 220-221.

Among these polymers, mention may be made of:
crosslinked or non-crosslinked, neutralized or non-neutralized copolymers, including from 15% to 60% by weight of AMPS units and from 40% to 85% by weight of (C₅-C₁₆)alkyl(meth)acrylamide or (C₅-C₁₆)alkyl(meth)acrylate units relative to the polymer, such as those described in patent application EP-A750899;
terpolymers including from 10 mol % to 90 mol % of acrylamide units, from 0.1 mol % to 10 mol % of AMPS units and from 5 mol % to 80 mol % of n-(C₆-C₁₈)alkylacrylamide units, such as those described in patent US-5089578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Among the cationic associative polymers, mention may be made of:

(a) cationic associative polyurethanes;

(b) the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

(c) quaternized (poly)hydroxyethylcelluloses modified with groups including at least one fatty chain, such as alkyl, arylalkyl or alkylaryl groups including at least 8 carbon atoms, or mixtures thereof. The alkyl radicals borne by the above quaternized celluloses or hydroxyethylcelluloses preferably include from 8 to 30 carbon atoms. The aryl radicals preferably denote phenyl, benzyl, naphthyl or anthryl groups. Examples of quaternized alkylhydroxyethylcelluloses containing C₈-C₃₀ fatty chains that may be indicated include the products Quatrisoft LM 200®, Quatrisoft LM-X 529-18-A®, Quatrisoft LM-X 529-18-B® (C₁₂ alkyl) and Quatrisoft LM-X 529-8® (C₁₈ alkyl) sold by the company Aqualon, and the products Crodacel QM®, Crodacel QL® (C₁₂ alkyl) and Crodacel QS® (C₁₈ alkyl) sold by the company Croda and the product Softcat SL 100® sold by the company Aqualon.

(d) cationic polyvinyllactam polymers.

Such polymers are described, for example, in patent application WO-00/68282.

As cationic poly(vinyllactam) polymers according to the invention, vinylpyrrolidone/dimethylaminopropylmethacrylamide/dodecyldimethylmethacrylam idopropylammonium tosylate terpolymers, vinylpyrrolidone/dimethylaminopropylmethacrylamide/cocoyldimethylmethacrylami dopropylammonium tosylate terpolymers, vinylpyrrolidone/dimethylaminopropylmethacrylamide/lauryldimethylmethacrylamid opropylammonium tosylate or chloride terpolymers are used notably.

The amphoteric associative polymers are preferably chosen from those including at least one noncyclic cationic unit. Even more particularly, those prepared from or comprising 1 to 20 mol %, preferably 1.5 to 15 mol % and even more particularly 1.5 to 6 mol % of fatty-chain monomer relative to the total number of moles of monomers are preferred.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:

(a) copolymers of vinylpyrrolidone and of fatty-chain hydrophobic monomers, of which examples that may be mentioned include:
the products Antaron V216® or Ganex V216® (vinylpyrrolidone/hexadecene copolymer) sold by the company ISP,
the products Antaron V220® or Ganex V220@(vinylpyrrolidone/eicosene copolymer) sold by the company ISP,

(b) copolymers of C₁-C₆ alkyl methacrylates or acrylates and of amphiphilic monomers including at least one fatty chain, for instance the oxyethylenated methyl acrylate/stearyl acrylate copolymer sold by the company Goldschmidt under the name Antil 208®.

(c) copolymers of hydrophilic methacrylates or acrylates and of hydrophobic monomers including at least one fatty chain, for instance the polyethylene glycol methacrylate/lauryl methacrylate copolymer;

(d) polyurethane polyethers including in their chain both hydrophilic blocks usually of polyoxyethylenated nature and hydrophobic blocks, which may be aliphatic sequences alone and/or cycloaliphatic and/or aromatic sequences;

(e) polymers with an aminoplast ether backbone containing at least one fatty chain, such as the Pure Thix® compounds sold by the company Sud-Chemie;

(f) celluloses or derivatives thereof, modified with groups comprising at least one fatty chain, such as alkyl, arylalkyl or alkylaryl groups or mixtures thereofin which the alkyl groups are of C₈, and in particular:
nonionic alkylhydroxyethylcelluloses such as the products Natrosol Plus Grade 330 CS and Polysurf 67 (C₁₆ alkyl) sold by the company Aqualon;
nonionic nonoxynylhydroxyethylcelluloses such as the product Amercell HM-1500 sold by the company Amerchol;
nonionic alkylcelluloses such as the product Bermocoll EHM 100 sold by the company Berol Nobel;

(g) associative guar derivatives, for instance hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers include at least two hydrocarbon-based lipophilic chains containing from 6 to 30 carbon atoms, separated by a hydrophilic block, the hydrocarbon-based chains possibly being side chains or chains at the end of the hydrophilic block. In particular, it is possible for one or more side chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The nonionic fatty-chain polyurethane polyethers may be triblock copolymers, the hydrophilic block of which is a polyoxyethylene chain including from 50 to 1000 oxyethylene groups. The nonionic polyurethane polyethers include a urethane bond between the hydrophilic blocks, hence the origin of the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, use may also be made of Rheolate 205® bearing a urea function, sold by the company Rheox, or Rheolate® 208, 204 or 212, and also Acrysol RM 184®.

Mention may also be made of the product Elfacos T210® bearing a C₁₂-C₁₄ alkyl chain, and the product Elfacos T212® bearing a C₁₈ alkyl chain, from Akzo.

The product DW 1206B® from Röhm & Haas bearing a C₂₀ alkyl chain and a urethane bond, provided at a solids content of 20% in water, may also be used.

Use may also be made of solutions or dispersions of these polymers, notably in water or in aqueous-alcoholic medium. Examples of such polymers that may be mentioned are Rheolate® 255, Rheolate® 278 and Rheolate® 244 sold by the company Rheox. Use may also be made of the products DW 1206F and DW 1206J sold by the company Röhm & Haas.

The polyurethane polyethers that may be used according to the invention are in particular those described in the article by G. Fonnum, J. Bakke and Fk. Hansen—Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold notably by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the cosmetic composition according to the invention comprises one or more thickening polymers; preferentially chosen from polysaccharides; more preferentially from celluloses, microbial gums such as xanthan gum, scleroglucan gum, mixtures thereof, these polysaccharides being optionally modified by a heat treatment, an esterification reaction, an etherification reaction, an amidation reaction or an oxidation reaction.

Even more preferentially, the polysaccharide(s) are chosen from cellulose ethers, which are preferably nonionic, microbial gums, in particular xanthan gum, scleroglucan gum.

Preferably, when the thickening polymer(s) are present in the composition according to the invention, the total content of thickening polymer(s) present is between 0.01% and 10% by weight, more preferentially between 0.05% and 8% by weight, even more preferentially between 0.1% and 5% by weight, even better still between 0.4% and 2% by weight, relative to the total weight of the composition.

The Silicones

The cosmetic composition according to the invention may also optionally comprise one or more silicones.

Preferably, the silicone(s) are chosen from polydialkylsiloxanes, notably polydimethylsiloxanes (PDMSs), and organomodified polysiloxanes including at least one functional group chosen from amine groups, oxyalkylene groups, aryl groups and alkoxy groups.

Organopolysiloxanes are defined in greater detail in Walter Noll's Chemistry and Technology of Silicones (1968), Academic Press. They may be volatile or non-volatile.

The non-volatile silicones that may be used in the composition according to the invention may preferably be non-volatile polydialkylsiloxanes, polyorganosiloxanes modified with organofunctional groups chosen from amine groups, aryl groups, oxyalkylene groups and alkoxy groups, and also mixtures thereof.

These silicones are more particularly chosen from polydialkylsiloxanes, among which mention may be made mainly of polydimethylsiloxanes bearing trimethylsilyl end groups. The viscosity of the silicones is measured at 25° C. according to ASTM standard 445 Appendix C.

Among these polydialkylsiloxanes, mention may be made, in a non-limiting manner, of the following commercial products:
the Silbione® oils of the 47 and 70047 series or the Mirasil® oils sold by Rhodia;
the oils of the Mirasil® series sold by the company Rhodia;
the oils of the 200 series from Dow Corning;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in accordance with the invention are silicones as defined above and including in their structure one or more organofunctional groups attached via a hydrocarbon-based group.

The organomodified silicones may be polydiarylsiloxanes, notably polydiphenylsiloxanes, polydialkylsiloxanes and polyalkylarylsiloxanes functionalized with the organofunctional groups mentioned previously.

The polyalkylarylsiloxanes are particularly chosen from polydimethyl/methylphenylsiloxanes and polydimethyl/diphenylsiloxanes that are linear and/or branched and that have a viscosity ranging from 1×10⁻⁵ to 5×10⁻² m²/s at 25° C.

Among these polyalkylarylsiloxanes, examples that may be mentioned include the products sold under the following names:
the Silbione® oils of the 70641 series from Rhodia;
oils of the Rhodorsil® 70633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may also be made of polyorganosiloxanes including:
substituted or unsubstituted amine groups, for instance the products sold under the names GP 4 Silicone Fluid and GP 7100 by Genesee. The substituted amine groups are in particular C₁-C₄ aminoalkyl groups;
alkoxylated groups, such as the product sold under the name Silicone Copolymer F-755 by SWS Silicones and Abil Wax® 2428, 2434 and 2440 by the company Goldschmidt;
oxyalkylene groups, in particular oxyethylene groups, such as the product sold under the name Xiameter OFX-0193 Fluid by the company Dow Corning.

The volatile silicones are more particularly chosen from those with a boiling point of between 60° C. and 260° C., and even more particularly from:
cyclic silicones including from 3 to 7 and preferably 4 to 6 silicon atoms.

These are, for example, octamethylcyclotetrasiloxane sold notably under the name Volatile Silicone 7207 by the company Union Carbide or Silbione 70045 V 2 by the company Rhodia, decamethylcyclopentasiloxane sold under the name Volatile Silicone 7158 by the company Union Carbide, and Silbione 70045 V 5 by the company Rhodia, and mixtures thereof.

Mention may also be made of cyclocopolymers of the dimethylsiloxane/methylalkylsiloxane type, such as Volatile Silicone FZ 3109 sold by the company Union Carbide, of chemical structure:

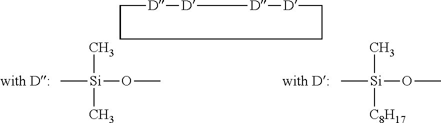

Mention may also be made of mixtures of cyclic silicones with organosilicon compounds, such as the mixture of octamethylcyclotetrasiloxane and tetrakis(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;
linear volatile silicones containing 2 to 9 silicon atoms and having a viscosity of less than or equal to 5×10⁻⁶ m²/s at 25° C. Examples include hexamethyldisiloxane, octamethyltrisiloxane and decamethyltetrasiloxane sold notably under the name SH 200 by Toray Silicone. Silicones belonging to this category are also described in the article published in Cosmetics and Toiletries, Vol. 91, January 76, pp. 27-32, Todd & Byers “Volatile Silicone Fluids for Cosmetics”. Preferably, the linear volatile silicones contain from 2 to 7 silicon atoms and better still from 3 to 6 silicon atoms.

Preferably, the volatile silicones are chosen from cyclic silicones including from 4 to 6 silicon atoms and linear silicones containing 4 to 6 silicon atoms.

Preferably, the non-volatile silicones are chosen from polydialkylsiloxanes, more particularly organomodified or non-organomodified polydimethylsiloxanes.

Preferably, the composition according to the invention comprises one or more silicones; more preferentially, the silicone(s) are chosen from polydimethylsiloxanes; polydimethylsiloxanes modified with organofunctional groups chosen from amine groups and oxyalkylene groups; and mixtures thereof.

According to a particular embodiment of the invention, the composition comprises one or more silicones chosen from polydimethylsiloxanes modified with one or more oxyalkylene groups.

According to another particular embodiment of the invention, the silicones are chosen from amino silicones.

The term “amino silicone” denotes any silicone including at least one primary, secondary or tertiary amine or a quaternary ammonium group.

The weight-average molecular masses of these amino silicones may be measured by gel permeation chromatography (GPC) at room temperature (25° C.), as polystyrene equivalent. The columns used are g styragel columns. The eluent is THF and the flow rate is 1 ml/min. 200 μl of a 0.5% by weight solution of silicone in THF are injected. Detection is performed by refractometry and UV-metry.

Preferably, the amino silicone(s) that may be used in the context of the invention are chosen from:

a) the polysiloxanes corresponding to formula (A):

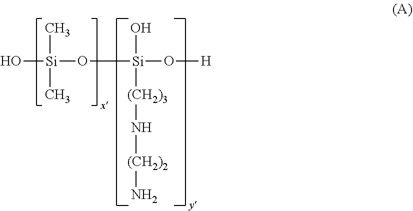

in which x′ and y′ are integers such that the weight-average molecular weight (Mw) is between 5000 and 500000 approximately;

b) the amino silicones corresponding to formula (B):

R′ₐG₃₋ₐ-Si(OSiG₂)ₙ-(OSiG_{b}R′_{2-b})ₘ—O-SiG₃₋ₐ-R′ₐ (B)

in which:
G, which may be identical or different, denotes a hydrogen atom or a phenyl, OH, C₁-C₈alkyl, for example methyl, or C₁-C₈alkoxy, for example methoxy, group,
a, which may be identical or different, denotes 0 or an integer from 1 to 3, in particular 0,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10;
R′, which may be identical or different, denotes a monovalent radical of formula —CqH2qL in which q is a number ranging from 2 to 8 and L is an optionally quaternized amine group chosen from the following groups:

—N(R″)₂; —N⁺(R″)₃A-; —NR″-Q-N(R″)₂ and —NR″-Q-N⁺(R″)₃A-,

in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A- represents a cosmetically acceptable anion, notably a halide such as fluoride, chloride, bromide or iodide.

Preferably, the amino silicones are chosen from the amino silicones of formula (B). Preferably, the amino silicones of formula (B) are chosen from the amino silicones corresponding to formulae (C), (D), (E), (F) and/or (G) below.

According to a first embodiment, the amino silicones corresponding to formula (B) are chosen from the silicones known as “trimethylsilyl amodimethicone” corresponding to formula (C):

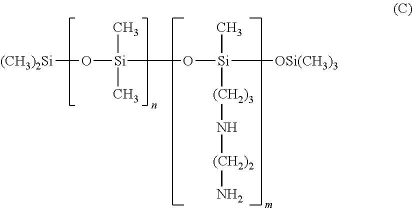

in which m and n are numbers such that the sum (n+m) ranges from 1 to 2000 and in particular from 50 to 150, it being possible for n to denote a number from 0 to 1999 and notably from 49 to 149, and for m to denote a number from 1 to 2000 and notably from 1 to 10.

According to a second embodiment, the amino silicones corresponding to formula (B) are chosen from the silicones of formula (D) below:

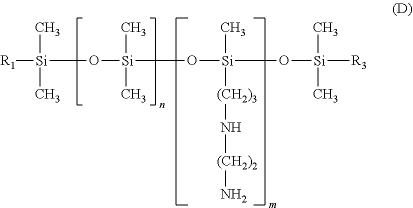

in which:
m and n are numbers such that the sum (n+m) ranges from 1 to 1000 and in particular from 50 to 250 and more particularly from 100 to 200; it being possible for n to denote a number from 0 to 999 and notably from 49 to 249 and more particularly from 125 to 175, and for m to denote a number from 1 to 1000 and notably from 1 to 10, and more particularly from 1 to 5;
R1, R2 and R3, which may be identical or different, represent a hydroxyl or C₁-C₄ alkoxy radical, at least one of the radicals R1 to R3 denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly equals 0.3:1.

The weight-average molecular mass (Mw) of these silicones preferably ranges from 2000 to 1000000 and more particularly from 3500 to 200000.

According to a third embodiment, the amino silicones corresponding to formula (B) are chosen from the silicones of formula (E) below:

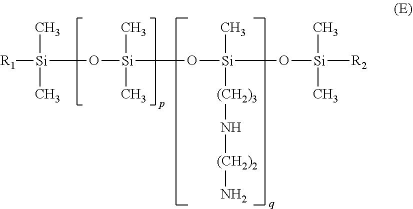

in which:
p and q are numbers such that the sum (p+q) ranges from 1 to 1000, in particular from 50 to 350 and more particularly from 150 to 250; it being possible for p to denote a number from 0 to 999 and notably from 49 to 349 and more particularly from 159 to 239, and for q to denote a number from 1 to 1000, notably from 1 to 10 and more particularly from 1 to 5;
R1 and R2, which are different, represent a hydroxyl or C₁-C₄ alkoxy radical, at least one of the radicals R1 or R2 denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly equals 1:0.95.

The weight-average molecular mass (Mw) of the silicone preferably ranges from 2000 to 200000, even more particularly from 5000 to 100000 and more particularly from 10000 to 50000.

The commercial products comprising silicones of structure (D) or (E) may include in their composition one or more other amino silicones the structure of which is different from formula (D) or (E).

A product containing amino silicones of structure (D) is sold by the company Wacker under the name Belsil® ADM 652.

A product containing amino silicones of structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The numerical mean size of the silicone particles in the emulsion generally ranges from 3 nm to 500 nanometres. Preferably, notably as amino silicones of formula (E), use is made of microemulsions with a mean particle size ranging from 5 nm to 60 nanometres (limits included) and more particularly from 10 nm to 50 nanometres (limits included). Thus, use may be made according to the invention of the amino silicone microemulsions of formula (E) sold under the names Finish CT 96 E® or SLM 28020® by the company Wacker.

According to a fourth embodiment, the amino silicones corresponding to formula (B) are chosen from the silicones of formula (F) below:

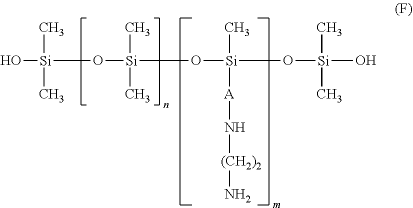

in which:
m and n are numbers such that the sum (n+m) ranges from 1 to 2000 and in particular from 50 to 150, it being possible for n to denote a number from 0 to 1999 and notably from 49 to 149, and for m to denote a number from 1 to 2000 and notably from 1 to 10;
A denotes a linear or branched alkylene radical containing from 4 to 8 carbon atoms and preferably 4 carbon atoms. This radical is preferably linear.

The weight-average molecular mass (Mw) of these amino silicones preferably ranges from 2000 to 1000000 and even more particularly from 3500 to 200000.

A silicone corresponding to this formula is, for example, the Xiameter MEM 8299 Emulsion from Dow Corning.

According to a fifth embodiment, the amino silicones corresponding to formula (B) are chosen from the silicones of formula (G) below:

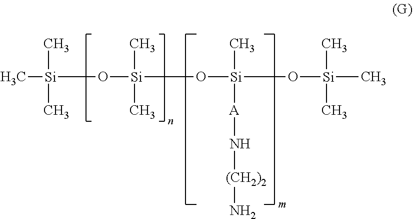

in which:
m and n are numbers such that the sum (n+m) ranges from 1 to 2000 and in particular from 50 to 150, it being possible for n to denote a number from 0 to 1999 and notably from 49 to 149, and for m to denote a number from 1 to 2000 and notably from 1 to 10;
A denotes a linear or branched alkylene radical containing from 4 to 8 carbon atoms and preferably 4 carbon atoms. This radical is preferably branched.

The weight-average molecular mass (Mw) of these amino silicones preferably ranges from 500 to 1000000 and even more particularly from 1000 to 200000.

A silicone corresponding to this formula is, for example, DC2-8566 Amino Fluid from Dow Corning;
c) the amino silicones corresponding to formula (H):

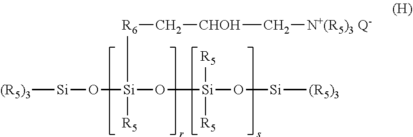

in which:
R₅ represents a monovalent hydrocarbon-based radical containing from 1 to 18 carbon atoms, and in particular a C₁-C₁₈ alkyl or C₂-C₁₈ alkenyl, for example methyl, radical;
R₆ represents a divalent hydrocarbon-based radical, notably a C₁-C₁₈ alkylene radical or a divalent C₁-C₁₈, for example C₁-C₈, alkyleneoxy radical linked to the Si via an SiC bond;
Q- is an anion such as a halide, notably chloride, ion or an organic acid salt, notably acetate;
r represents a mean statistical value ranging from 2 to 20 and in particular from 2 to 8;
s represents a mean statistical value ranging from 20 to 200 and in particular from 20 to 50.

Such amino silicones are notably described in patent U.S. Pat. No. 4,185,087;

d) the silicones comprising a quaternary ammonium having the formula below:

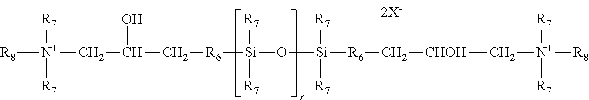

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon-based radical, notably a C₁-C₁₈alkylene radical or a divalent C₁-C₁₈, for example C₁-C₈, alkyleneoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X— is an anion such as a halide, notably chloride, ion or an organic acid salt, notably acetate;
r represents a mean statistical value ranging from 2 to 200 and in particular from 5 to 100.

These silicones are described, for example, in patent application EP-A 0530 974;

e) the amino silicones of formula (J):

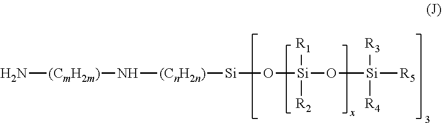

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;

f) the multiblock polyoxyalkylenated amino silicones, of the type (AB)n, A being a polysiloxane block and B being a polyoxyalkylenated block including at least one amine group.

Said silicones are preferably constituted of repeating units of the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10000 and more particularly from 10 to 5000;
R″ is a hydrogen atom or a methyl;
R, which may be identical or different, represent a linear or branched divalent C₂-C₁₂ hydrocarbon-based radical, optionally including one or more heteroatoms such as oxygen; preferably, R denotes an ethylene radical, a linear or branched propylene radical, a linear or branched butylene radical or a radical —CH₂CH₂CH₂OCH₂CH(OH)CH₂—; preferentially, R denotes a radical —CH₂CH₂CH₂OCH₂CH(OH)CH₂—;
R′, which may be identical or different, represent a linear or branched divalent C₂-C₁₂ hydrocarbon-based radical, optionally including one or more heteroatoms such as oxygen; preferably, R′ denotes an ethylene radical, a linear or branched propylene radical, a linear or branched butylene radical or a radical —CH₂CH₂CH₂OCH₂CH(OH)CH₂—; preferentially, R′ denotes —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1000000 and more particularly between 10000 and 200000.

Mention may be made notably of the silicones sold under the names Silsoft A-843 or Silsoft A+ by Momentive;

g) and mixtures thereof.

According to a preferred embodiment of the invention, the cosmetic composition comprises one or more silicones; preferentially chosen from the amino silicones as described previously, the polydimethylsiloxanes as described previously, polydimethylsiloxanes modified with organofunctional groups chosen from amine groups and oxyalkylene groups as described previously, and mixtures thereof.

Even more preferentially according to this embodiment of the invention, the silicone(s) are chosen from polydimethylsiloxanes; polydimethylsiloxanes modified with organofunctional groups chosen from amine groups and oxyalkylene groups; and mixtures thereof.

Even better still according to this embodiment of the invention, the silicone(s) are chosen from polydimethylsiloxanes modified with one or more oxyalkylene groups.

Preferably, when the composition according to the invention comprises one or more silicones, the total content of silicone(s) is between 0.01% and 15% by weight, more preferentially between 0.05% and 10% by weight, even more preferentially between 0.1% and 5% by weight, relative to the total weight of the composition.

The Alkaline Agents

The cosmetic composition according to the invention may optionally also comprise one or more alkaline agents.

Preferably, the alkaline agent(s) may be chosen from organic alkaline agents and inorganic alkaline agents.

The organic alkaline agent(s) are preferably chosen from organic amines with a pK_{b} at 25° C. of less than 12, more preferentially less than 10 and even more advantageously less than 6. It should be noted that it is the pK_{b} corresponding to the function of highest basicity. In addition, the organic amines do not comprise any alkyl or alkenyl fatty chain comprising more than ten carbon atoms.

The organic alkaline agent(s) are preferably chosen from alkanolamines, in particular mono-, di- or trihydroxy(C₁-C₆)alkylamines, such as 2-amino-2-methylpropanol, oxyethylenated and/or oxypropylenated ethylenediamines, amino acids, the polyamines of formula (VII) below, and mixtures thereof:

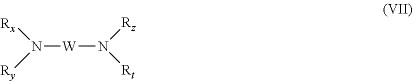

in which formula (VII) W is a divalent C₁ to C₆ alkylene radical optionally substituted with one or more hydroxyl groups or a C₁ to C₆ alkyl radical, and/or optionally interrupted with one or more heteroatoms such as O, or NRᵤ; Rₓ, R_{y}, R_{z}, Rₜ, and Rᵤ, which may be identical or different, represent a hydrogen atom, or a C₁ to C₆ alkyl or C₁ to C₆ hydroxyalkyl or C₁ to C₆ aminoalkyl radical.

Examples of amines of formula (VII) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

As amino acids that may be used in the present invention, mention may notably be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (VIII) below, and also salts thereof:

R—CH₂—CH(NH₂)—C(O)—OH (VIII)

in which formula (VIII) R represents a group chosen from imidazolyl, preferably imidazolyl-4-yl; aminopropyl; aminoethyl; —(CH₂)₂N(H)—C(O)—NH₂; and —(CH₂)₂—N(H)—C(NH)—NH₂.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that may be used in the present invention, mention may be made notably of carnosine, anserine and balenine.

The organic amine may also be chosen from compounds including a guanidine function. As amines of this type that may be used in the present invention, besides arginine, which has already been mentioned as an amino acid, mention may be made notably of creatine, creatinine, 1,1-dimethylguanidine, 1,1-diethylguanidine, glycocyamine, metformin, agmatine, n-amidinoalanine, 3-guanidinopropionic acid, 4-guanidinobutyric acid and 2-([amino(imino)methyl]amino)ethane-1-sulfonic acid.

Among the inorganic alkaline agents that may be used in the process according to the invention, mention may be made of mineral hydroxides.

The mineral hydroxides may be chosen from alkali metal, alkaline-earth metal and transition metal hydroxides. Examples of mineral hydroxides that may be mentioned include sodium hydroxide, lithium hydroxide, calcium hydroxide, magnesium hydroxide, barium hydroxide, strontium hydroxide, manganese hydroxide and zinc hydroxide.

Among the mineral hydroxides, sodium hydroxide is preferred.

The preferred hydroxide compounds are sodium hydroxide, calcium hydroxide and lithium hydroxide.

Preferably, the alkaline agent(s) that are useful in the invention are chosen from aqueous ammonia, alkanolamines, amino acids in neutral or ionic form, in particular basic amino acids, and preferably corresponding to those of formula (VIII).

According to a preferred embodiment of the invention, the cosmetic composition comprises one or more alkaline agents; more preferentially, the alkaline agent(s) are chosen from monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane, and mixtures thereof; even more preferentially, the alkaline agent is 2-amino-2-methyl-1-propanol.

Preferably, when the organic alkaline agent(s) are present in the composition, the total content of alkaline agent(s) is between 0.1% and 10% by weight, more preferentially between 0.3% and 10% by weight, and even more preferentially between 0.4% and 5% by weight, relative to the total weight of the composition.

Non-Silicone Fatty Substances

The cosmetic composition according to the invention may also optionally comprise one or more non-silicone fatty substances.

The term “fatty substance” means an organic compound that is insoluble in water at ordinary temperature (25° C.) and at atmospheric pressure (760 mmHg or 1.013×10⁵ Pa) (solubility of less than 5%, preferably of less than 1% and even more preferentially of less than 0.1% by weight). The non-silicone fatty substances (i.e. the fatty substances not comprising any silicon atoms in their structure) have in their structure at least one hydrocarbon-based chain including at least 6 carbon atoms. In addition, the non-silicone fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The non-silicone fatty substances of the invention do not contain any salified carboxylic acid groups.

In addition, the non-silicone fatty substances of the invention are not (poly)oxyalkylenated or (poly)glycerolated ethers.

Preferably, the non-silicone fatty substance(s) are chosen from liquid non-silicone fatty substances, solid non-silicone fatty substances, and mixtures thereof.

The term “liquid fatty substance” or “oil” means a “fatty substance” that is liquid at room temperature (25° C.) and at atmospheric pressure (760 mmHg or 1.013×10⁵ Pa).

The term “solid fatty substance” means a “fatty substance” that is solid at room temperature (25° C.) and at atmospheric pressure (760 mmHg or 1.013×10⁵ Pa).

Liquid Non-Silicone Fatty Substances

The composition used in the process according to the invention may comprise one or more liquid non-silicone fatty substances. These agents may be chosen notably from liquid fatty alcohols; mineral, plant or animal oils; liquid fatty esters; liquid hydrocarbons, and mixtures thereof.

The liquid fatty alcohols may be linear or branched; they preferably comprise 8 to 30 carbon atoms; they may be saturated or unsaturated.

The saturated liquid fatty alcohols are preferably branched. They may optionally comprise in their structure at least one aromatic or non-aromatic ring. Preferably, they are acyclic. More particularly, the saturated liquid fatty alcohols are chosen from octyldodecanol, isostearyl alcohol, 2-hexyldecanol, and also palmityl, myristyl, stearyl and lauryl alcohols, and mixtures thereof.

The unsaturated liquid fatty alcohols contain in their structure at least one double or triple bond, and preferably one or more double bonds. When several double bonds are present, there are preferably 2 or 3 of them, and they may be conjugated or unconjugated. They may optionally comprise in their structure at least one aromatic or non-aromatic ring. Preferably, they are acyclic. More particularly, the unsaturated liquid fatty alcohols are chosen from oleyl alcohol, linoleyl alcohol, linolenyl alcohol and undecylenyl alcohol, and mixtures thereof.

Among the mineral, plant or animal oils that may be used, mention may be made notably, as oils of plant origin, of sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, corn oil, hazelnut oil, shea butter, palm oil, apricot kernel oil, beauty-leaf oil, evening primrose oil or camelina oil; as oil of animal origin, perhydrosqualene; as oils of mineral origin, liquid paraffin and liquid petroleum jelly; and mixtures thereof.

The liquid fatty esters may be esters of monoalcohols or of polyols with monoacids or polyacids, at least one of the alcohols and/or acids including at least one chain of more than 7 carbon atoms. Preferably, the liquid fatty ester according to the invention is chosen from esters of a fatty acid and of a monoalcohol. Preferably, at least one of the alcohols and/or acids is branched. Mention may be made of isopropyl myristate, isopropyl palmitate, isononyl or isostearyl isononanoate, 2-ethylhexyl palmitate, 2-hexyldecyl laurate, 2-octyldecyl palmitate and 2-octyldodecyl myristate, purcellin oil (stearyl octanoate), isopropyl lanolate, and mixtures thereof.

The term “liquid hydrocarbon” means a hydrocarbon composed solely of carbon and hydrogen atoms, which is liquid at 25° C. and 1 atm, which is notably of mineral or plant origin, preferably of plant origin.

As liquid hydrocarbon that may be used in the composition according to the invention, mention may be made of:
linear or branched, optionally cyclic, C₆-C₁₆ alkanes; mention may be made of hexane, undecane, dodecane, tridecane, and isoparaffins, for instance isohexadecane, isododecane and isodecane;
linear or branched hydrocarbons notably of mineral, animal or synthetic origin with more than 16 carbon atoms, such as volatile or non-volatile liquid paraffins, petroleum jelly, liquid petroleum jelly, polydecenes, hydrogenated polyisobutene such as the product sold under the brand name Parleam® by the company NOF Corporation, and squalane.

Preferably, when the cosmetic composition according to the invention comprises one or more non-silicone liquid fatty substances, it comprises them in a total amount ranging from 0.1% to 15% by weight, preferably from 0.5% to 10% by weight and even better still from 1% to 5% by weight, relative to the total weight of the cosmetic composition.

Non-Silicone Solid Fatty Substances

The composition used in the process according to the invention may comprise one or more non-silicone solid fatty substances. These substances may be chosen notably from solid fatty alcohols; solid fatty esters, ceramides; animal, plant or mineral waxes other than ceramides; and mixtures thereof.

The solid fatty alcohols that may be used are preferably chosen from saturated or unsaturated, linear or branched, preferably linear and saturated, (mono)alcohols including from 8 to 30 carbon atoms and notably 10 to 24 carbon atoms. Mention may be made, for example, of cetyl alcohol, stearyl alcohol and the mixture thereof (cetylstearyl alcohol).

The solid fatty esters that may be used are preferably chosen from esters derived from C₉-C₂₆ monocarboxylic acids and from C₉-C₂₆ alcohols. Mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used. Mention may be made notably of diethyl sebacate, diisopropyl sebacate, diisopropyl adipate, di-n-propyl adipate, dioctyl adipate and dioctyl maleate.

Preferentially, it is preferred to use C₉-C₂₆ alkyl palmitates, notably myristyl, cetyl or stearyl palmitates, and C₉-C₂₆ alkyl myristates such as cetyl myristate, stearyl myristate and myristyl myristate or mixtures of myristyl palmitate and myristyl stearate.

The ceramides, or ceramide analogues such as glycoceramides, that may be used in the composition according to the invention, are known per se; mention may in particular be made of ceramides of classes I, II, III and V according to the Dawning classification; they are molecules which may correspond to the formula below:

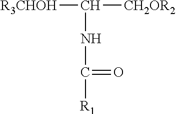

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom, a (glycosyl)n group, a (galactosyl)m group or a sulfogalactosyl group, in which n is an integer ranging from 1 to 4 and m is an integer ranging from 1 to 8;
R₃ denotes a C₁₅-C₂₆ hydrocarbon-based group, which is saturated or unsaturated in the alpha position, this group possibly being substituted with one or more C₁-C₁₄ alkyl groups;
it being understood that, in the case of natural ceramides or glycoceramides, R₃ may also denote a C₁₅-C₂₆ alpha-hydroxyalkyl group, the hydroxyl group optionally being esterified with a C₁₆-C₃₀ alpha-hydroxy acid.

The ceramides more particularly preferred are the compounds for which R₁ denotes a saturated or unsaturated alkyl derived from C₁₆-C₂₂ fatty acids; R₂ denotes a hydrogen atom and R₃ denotes a saturated linear C₁₅ group.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

Use may also be made of the compounds for which R₁ denotes a saturated or unsaturated alkyl radical derived from C₁₂-C₂₂ fatty acids; R₂ denotes a galactosyl or sulfogalactosyl radical; and R₃ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine; 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof.

For the purposes of the present invention, a wax is a lipophilic compound, which is solid at room temperature (25° C.), with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but, on returning the temperature of the mixture to room temperature, recrystallization of the wax, which is microscopically and macroscopically detectable (opalescence), is obtained in the oils of the mixture.

As waxes, other than the ceramides above, that can be used in the present invention, mention may be made of waxes of animal origin, such as beeswaxes or modified beeswaxes (cera bellina), spermaceti, lanolin wax and lanolin derivatives; plant waxes such as carnauba wax, candelilla wax, esparto wax, ouricury wax, Japan wax, cocoa butter, cork-fibre wax, sugarcane wax, olive-tree wax, rice wax, hydrogenated jojoba wax, absolute waxes of flowers; mineral waxes, for example paraffin wax, petroleum jelly wax, lignite wax, microcrystalline waxes, ozokerites, and mixtures thereof.

Preferably, the non-silicone solid fatty substances may be chosen from solid fatty alcohols and solid fatty esters.

Preferably, when the cosmetic composition according to the invention comprises one or more solid fatty substances, it comprises them in a total amount ranging from 0.1% to 15% by weight, preferably from 0.5% to 10% by weight and even better still from 1% to 5% by weight, relative to the total weight of the cosmetic composition.

According to a preferred embodiment of the invention, the cosmetic composition also comprises one or more non-silicone fatty substances; more preferentially, the non-silicone fatty substance(s) are chosen from the solid non-silicone fatty substances as described previously; even more preferentially from the solid fatty alcohols as described previously, notably cetearyl alcohol, stearyl alcohol, cetyl alcohol, and mixtures thereof.

Preferably, when the non-silicone fatty substance(s) are present in the cosmetic composition according to the invention, the total content of non-silicone fatty substance(s) is between 0.01% and 20% by weight, more preferentially between 0.1% and 15% by weight, and even more preferentially between 0.5% and 10% by weight, relative to the total weight of the composition.

Preferably, when the solid fatty alcohol(s) are present in the cosmetic composition according to the invention, the total content of solid fatty alcohol(s) is between 0.01% and 20% by weight, more preferentially between 0.1% and 15% by weight, and even more preferentially between 0.5% and 10% by weight, relative to the total weight of the composition.

According to the invention, the cosmetic composition is aqueous.

Preferably, the amount of water may range from 5% to 98% by weight, better still from 15% by weight to 95% by weight, even better still from 25% to 93% by weight and even more preferentially from 50% to 90% by weight, relative to the total weight of the composition.

The aqueous cosmetic composition may optionally also comprise one or more organic solvents.

Examples of organic solvents that may notably be used include those that are liquid at 25° C. and 1.013×10⁵ Pa and that are notably water-soluble, such as C₁-C₇ alcohols, notably C₁-C₇ aliphatic or aromatic monoalcohols, and C₃-C₇ polyols and C₃-C₇ polyol ethers, which may thus be used as a mixture with water. Advantageously, the organic solvent may be chosen from ethanol and isopropanol, and mixtures thereof.

The pH of the cosmetic composition is between 2.5 and 6.

Preferably, the pH of the cosmetic composition is between 3 and 5, even better still between 3 and 4.

The pH of these compositions may be adjusted to the desired value by means of basifying agents as described previously or of acidifying agents. Among the acidifying agents, examples that may be mentioned include mineral or organic acids, for instance hydrochloric acid or orthophosphoric acid, carboxylic acids, for instance acetic acid, tartaric acid, citric acid and lactic acid, and sulfonic acids.

The composition according to the invention may also contain additives usually used in cosmetics, such as preserving agents, fragrances and colourants.

These additives may be present in the composition according to the invention in an amount ranging from 0 to 20% by weight, relative to the total weight of the composition.

A person skilled in the art will take care to select these optional additives and amounts thereof so that they do not harm the properties of the processes of the present invention.

The cosmetic composition may be in the form of a lotion, a gel, a milk or a cream.

According to a first preferred embodiment of the invention, step i) of the process according to the invention is performed before step ii).

More preferentially according to this embodiment, the cosmetic composition is liquid (at 25° C. and at atmospheric pressure); even more preferentially, the viscosity of said composition is less than 500 mPa·s (i.e. 500 cP), better still less than 250 mPa·s (i.e. 250 cP) and even better still less than 100 mPa·s (i.e. 100 cP).

According to a second preferred embodiment of the invention, step ii) of the process according to the invention is performed before step i).

More preferentially according to this embodiment, the cosmetic composition is in thickened form, in particular in the form of a cream or a gel; even more preferentially, the viscosity of said composition is greater than 500 mPa·s (i.e. 500 cP), preferentially between 550 and 2000 mPa·s (i.e. between 550 and 2000 cP).

According to this embodiment, the composition according to the invention comprises one or more thickening polymers as described previously, preferably chosen from polysaccharides, preferentially from cellulose ethers, in particular nonionic cellulose ethers, and microbial gums, in particular xanthan gum, scleroglucan gum.

The viscosity of the compositions according to the invention may be measured using a rheometer such as a Rheomat RM180 machine at 25° C. and at a spin speed of 200 rpm, the measurement being taken after 30 seconds of rotation.

Heating of the Keratin Fibres

The process according to the invention comprises at least one step of heating said fibres to a temperature of greater than or equal to 35° C.

Preferably, during step iii) of the process according to the invention, the keratin fibres are heated to a temperature of between 35 and 210° C., more preferentially between 35° C. and 100° C., even more preferentially between 40 and 80° C.

Step iii) of heating the keratin fibres to a temperature of between 40 and 80° C. is very preferred, this temperature range making it possible most particularly to promote the reduction of the damage caused to the keratin fibres, and also the preservation of their integrity and of their mechanical properties.

Preferably, step iii) of heating the keratin fibres lasts for between 10 and 45 minutes; more preferentially between 15 and 30 minutes.

Preferably, during step iii) of the process according to the invention, the keratin fibres are heated using one or more heating devices chosen from a hairdryer, a heating lamp, notably an infrared lamp such as a Climazon, a curling iron, a heating hood, heated curlers; more preferentially from a heating hood, a heating lamp, notably an infrared lamp such as a Climazon, and heated curlers.

For the purposes of the invention, said heated curlers may be the curlers used as curling means as described previously.

For the purposes of the invention, when the keratin fibre curling means used in step i) of the process is (are) chosen from heated curlers, said heated curler(s) are at room temperature (i.e. between 15 and 30° C.) and only heat said fibres once step i) and step ii) have been performed.

According to a preferred embodiment of the process according to the invention, there is no step of rinsing said keratin fibres between the implementation of step ii) and the implementation of step iii).

In other words, according to this embodiment of the process, the heating step iii) is performed while the aqueous cosmetic composition is present on the keratin fibres.

Preferably, according to this embodiment, the heating step iii) is performed while the aqueous cosmetic composition is present on the keratin fibres rolled up on one or more keratin fibre curling means.

The process according to the invention may optionally comprise a step iv) of covering the keratin fibres using a bonnet. Step iv) is preferably performed after steps i) and ii), and before step iii).

According to a particular embodiment, the process according to the invention comprises a step v) of applying to the keratin fibres an oxidizing composition comprising one or more chemical oxidizing agents, performed after step iii). More preferentially, said oxidizing composition comprises hydrogen peroxide and/or persalts.

Preferably, the process according to the invention comprises a step vi) of rinsing the keratin fibres, performed after step iii), and before the optional step v).

More particularly, the process according to the invention is performed on keratin fibres which have, prior to step i) and to step ii), been dyed and/or lightened.

A subject of the invention is also the use of the cosmetic composition as defined previously for curling keratin fibres, in particular human keratin fibres such as the hair, which have been dyed and/or lightened beforehand.

The examples that follow serve to illustrate the invention without, however, being limiting in nature.

### EXAMPLES

### Example 1

The cosmetic composition A that may be used in the process according to the invention is prepared from the ingredients shown in the table below, the amounts of which are expressed as weight percentages of active material (AM).

| | A |
|---|---|
| INGREDIENTS | (invention) |
| Thiolactic acid | 8 |
| PEG-12 Dimethicone | 2 |
| Polysorbate 20 | 1 |
| 2-Amino-2-methylpropan-1-ol | 2 |
| Water | qs 100 |
| pH | 3.5 ± 0.2 |

### Tensile Test:

2.7 g locks of hair 26 cm long, which have been highly sensitized by bleaching, washed beforehand with a standard shampoo (Pro-Classique from L'Oreal Professionnel), are wound while wet onto curlers.
The locks are then treated according to processes 1 and 2 below:

### Process 1 (Invention):

In the following order:
Application of composition A according to the invention, in a proportion of 5.4 g of composition per lock,
Heating of the locks for 15 minutes at a temperature of 40° C. (Climazon infrared lamp), and the locks are covered with a stretchable plastic film,
Rinsing of the locks with water,
Application of an oxidizing composition comprising 2.4% by weight of H₂O₂, relative to the total weight of the oxidizing composition. The locks are then left to stand for 10 minutes at room temperature (25° C.),
Rinsing of the locks with water,
Natural drying.

### Process 2 (Comparative):

In the following order:
Application of a conventional permanent-waving reducing composition (notably comprising 6.7% by weight of ammonium thioglycolate and 0.01% by weight of cysteine, relative to the total weight of the composition, pH 8.5±0.2), in a proportion of 5.4 g of composition per lock,
The locks are left to stand for 10 minutes at room temperature (25° C.),
Rinsing of the locks with water,
Application of an oxidizing composition comprising 2.4% by weight of H₂O₂, relative to the total weight of the oxidizing composition. The locks are then left to stand for 10 minutes at room temperature (25° C.),
Rinsing of the locks with water,
Natural drying.

On conclusion of the various processes and removal of the curlers, 33 hair strands are taken from each of the locks thus treated with composition A or the comparative composition. 33 hair strands are also taken from a lock of untreated hair which has been highly sensitized by bleaching.

Tensile tests are then performed on each of the fibres: the tensile strength is measured during the elongation of the fibre to the point of rupture, using a Dia-stron tensile testing machine. The breaking strength of the hair is then calculated. The higher the breaking strength, the more resistant the hair is to breaking, and thus the more its integrity is conserved.

The mean breaking strength values (MPa) are given below:

| Protocol | Breaking strength (MPa) |
|---|---|
| Control | 118.5 ± 9.9 |
| Process 1 (invention) | 110.3 ± 7.2 |
| Process 2 (comparative) | 80.3 ± 4.6 |

It is observed that process 1 according to the invention leads to higher breaking strengths, and thus to better conservation of the integrity of the hair, relative to the comparative process 2.

Other examples of cosmetic compositions that may be used in the process according to the invention were prepared from the ingredients shown in the table below, the amounts of which are expressed as weight percentages of active material (AM).

| | B |
|---|---|
| INGREDIENTS | (invention) |
| Thiolactic acid | 8 |
| Dimethicone and amodimethicone | 2 |
| Steareth-20 | 1 |
| Cetearyl alcohol | 5 |
| Behentrimonium chloride | 3.2 |
| Scleroglucan gum | 0.46 |
| Xanthan gum | 0.15 |
| 2-Amino-2-methylpropan-1-ol | 3 |
| Water | qs 100 |
| pH | 3.5 ± 0.2 |

| | C |
|---|---|
| INGREDIENTS | (invention) |
| Thiolactic acid | 8 |
| PEG-12 Dimethicone | 2 |
| Polysorbate 20 | 1 |
| Hydroxyethylcellulose | 0.75 |
| 2-Amino-2-methylpropan-1-ol | 2 |
| Water | qs 100 |
| pH | 3.5 ± 0.2 |

### Example 2

The cosmetic compositions A and B are prepared from the ingredients shown in the table below, the amounts of which are expressed as weight percentages of active material (AM).

| | A | B |
|---|---|---|
| INGREDIENTS | (invention) | (Comparative) |
| Thiolactic acid | 8 | — |
| Thioglycolic acid | — | 8 |
| PEG-12 Dimethicone | 2 | 2 |
| Polysorbate 20 | 1 | 1 |
| 2-Amino-2-methylpropan-1-ol | 2 | 2 |
| Water | qs 100 | qs 100 |
| pH | 3.5 ± 0.2 | 3.5 ± 0.2 |

2.7 g locks of hair 26 cm long, which have been highly sensitized by bleaching, washed beforehand with a standard shampoo (Pro-Classique from L'Oreal Professionnel), are wound while wet onto curlers according to a standardised protocol.

The locks are then treated according to the following process:
Application of respectively composition A or composition B, in a proportion of 5.4 g of composition per lock,
Heating of the locks for 15 minutes at a temperature of 40° C. (Climazon infrared lamp), and the locks are covered with a stretchable plastic film,
Rinsing of the locks with water,
Application of an oxidizing composition comprising 2.4% by weight of H₂O₂, relative to the total weight of the oxidizing composition.
The locks are then left to stand for 10 minutes at room temperature (25° C.),
Rinsing of the locks with water and wrung out.

In order to evaluate the preservation of the integrity of the fibre, the elasticity of the wet hair is evaluated by stretching it on both sides: the more elastic it is, the more it returns to its original shape and its integrity is preserved.

If the hair breaks after stretching, it is no longer elastic and its integrity has not been preserved.

The results performed by a trained expert are given below:

| A | B |
|---|---|
| (invention) | (Comparative) |
| The hair is elastic, it returns | The hair breaks quickly |
| to its original shape | |
| Integrity is conserved | Integrity is not conserved |

We can observe that the locks treated with the composition A (invention) present a better conservation of their integrity than the locks treated with the composition B (comparative).

1. Process for curling keratin fibres, in particular human keratin fibres such as the hair, comprising at least the following steps:
i) rolling said fibres around one or more keratin fibre curling means;
ii) applying to said fibres an aqueous cosmetic composition:
a. comprising at least one reducing agent chosen from thiolactic acid, salts thereof, and mixtures thereof, and
b. with a pH between 2.5 and 6;
iii) heating said fibres to a temperature of greater than or equal to 35° C.; it being understood that step iii) is performed after steps i) and ii).

2. Process according to the preceding claim, characterized in that there is no step of rinsing said keratin fibres between the implementation of step ii) and the implementation of step iii).

3. Process according to either of the preceding claims, characterized in that the curling means are chosen from curlers and heated curlers.

4. Process according to any one of the preceding claims, characterized in that the content of reducing agent(s) chosen from thiolactic acid, salts thereof, and mixtures thereof, is between 1% and 15% by weight, preferably between 2% and 13% by weight, more preferentially between 3% and 12%, and even more preferentially between 4% and 10% by weight, relative to the total weight of the composition.

5. Process according to any one of the preceding claims, characterized in that said composition also comprises one or more additional agents chosen from one or more nonionic surfactants, one or more cationic surfactants, one or more anionic surfactants, one or more thickening polymers, one or more silicones, one or more alkaline agents, and/or one or more non-silicone fatty substances.

6. Process according to the preceding claim, characterized in that:
the total content of nonionic surfactant(s) is between 0.01% and 20% by weight, better still between 0.1% and 15% by weight, even better still between 0.2% and 10% by weight, or even between 0.5% and 6% by weight, relative to the total weight of the composition; and/or
the total content of cationic surfactant(s) is between 0.1% and 10% by weight, better still between 0.5% and 8% by weight, even better still between 1% and 5% by weight, relative to the total weight of the composition; and/or
the total content of anionic surfactant(s) is between 0.10% and 10% by weight, better still between 0.5% and 8% by weight, even better still between 1% and 5% by weight, relative to the total weight of the composition; and/or
the total content of thickening polymer(s) is between 0.01% and 10% by weight, better still between 0.05% and 8% by weight, even better still between 0.1% and 5% by weight, or even between 0.4% and 2% by weight, relative to the total weight of the composition; and/or
the total content of silicone(s) is between 0.01% and 15% by weight, better still between 0.05% and 10% by weight, even better still between 0.1% and 5% by weight, relative to the total weight of the composition; and/or
the total content of alkaline agent(s) is between 0.1% and 10% by weight, better still between 0.3% and 10% by weight and even better still between 0.4% and 5% by weight, relative to the total weight of the composition; and/or
the total content of fatty substance(s) is between 0.010% and 20% by weight, better still between 0.1% and 15% by weight, even better still between 0.5% and 10% by weight, relative to the total weight of the composition.

7. Process according to any one of the preceding claims, characterized in that the pH of said composition between 3 and 5, preferentially between 3 and 4.

8. Process according to any one of the preceding claims, characterized in that step i) is performed before step ii); preferably, said composition is liquid; more preferentially, the viscosity of said composition is less than 500 mPa·s, better still less than 250 mPa·s, even better still less than 100 mPa·s.

9. Process according to any one of claims 1 to 7, characterized in that step ii) is performed before step i); preferably, said composition is in thickened form; more preferentially in the form of a cream or a gel; even more preferentially, the viscosity of said composition is greater than 500 mPa·s, even better still between 550 and 2000 mPa·s.

10. Process according to any one of the preceding claims, characterized in that said fibres are heated to a temperature of between 35 and 210° C.; preferably between 35 and 100° C. and more preferentially between 40 and 80° C.

11. Process according to any one of the preceding claims, characterized in that step iii) of heating said fibres lasts for between 10 and 45 minutes; preferably between 15 and 30 minutes.

12. Process according to any one of the preceding claims, characterized in that it comprises a step v) of applying to said fibres an oxidizing composition comprising one or more chemical oxidizing agents, performed after step iii); preferably, said oxidizing composition comprises hydrogen peroxide and/or persalts.

13. Process according to any one of the preceding claims, characterized in that it comprises a step vi) of rinsing said keratin fibres, performed after step iii), and before the optional step v).

14. Use of the cosmetic composition as defined in any one of claims 1 and 4 to 7 for curling keratin fibres, in particular human keratin fibres such as the hair, which have been dyed and/or lightened beforehand.
